SEQUENCE LISTING (4 sequences)

SEQ ID NO: 1 - DNA, 23 bp, Artificial Sequence
  Primer
cagcggcacaggtcgaacaccgg

SEQ ID NO: 2 - DNA, 21 bp, Artificial Sequence
  Primer
aaaccagacttcacgctcggc

SEQ ID NO: 3 - DNA, 20 bp, Artificial Sequence
  Primer
gagggtttgcaggtccagag

SEQ ID NO: 4 - DNA, 18 bp, Artificial Sequence
  Primer
acatgctggggtaggtgc

(19) United States
(12) Patent Application Publication
(10) Pub. No.: US 2023/0213531 A1
(43) Pub. Date: Jul. 6, 2023

(54) ANALYTIC METHOD AND KIT FOR DIAGNOSING RECURRENT MISCARRIAGE

(71) Applicant: CHA UNIVERSITY INDUSTRY-ACADEMIC COOPERATION FOUNDATION, Pocheon-si, Gyeonggi-do (KR)

(72) Inventors: Kwang-Hyun BAEK, Seoul (KR); Chang-Zhu PEI, Seongnam-si, Gyeonggi-do (KR)

(73) Assignee: CHA UNIVERSITY INDUSTRY-ACADEMIC COOPERATION FOUNDATION, Pocheon-si, Gyeonggi-do (KR)

(21) Appl. No.: 17/905,364

(22) PCT Filed: Feb. 26, 2021
(86) PCT No.: PCT/KR2021/002429

§ 371 Date: Aug. 31, 2022

(30) Foreign Application Priority Data
Mar. 3, 2020 (KR) 10-2020-0026269

(51) Int. Cl. G01N 33/68; C12Q 1/6837; C12Q 1/6883

(52) U.S. Cl. CPC G01N 33/689; C12Q 1/6837; C12Q 1/6883; C12Q 2600/158; G01N 2800/368

(57) ABSTRACT

Provided are an analytical method for providing information necessary for diagnosing recurrent pregnancy loss, comprising measuring an expression level of HtrA4 protein or an expression level of a gene encoding the same in a subject’s sample, and a kit for diagnosing recurrent pregnancy loss, comprising a molecule capable of measuring an expression level of HtrA4 protein or an expression level of a gene encoding the same

### TECHNICAL FIELD

The present invention relates to an analytical method for providing information necessary for diagnosing recurrent pregnancy loss and a kit for diagnosing recurrent pregnancy loss.

### BACKGROUND ART

As women’s first marriage age increases due to an increase in women’s social advancement in modern society unlike the past, the proportion of various infertility-related diseases, including recurrent pregnancy loss, is also steadily increasing. And, because of the psychological pain caused by the pressure having to carry on a family line, recurrent pregnancy loss often leads to family breakdown, exacerbating serious social problems. Recurrent pregnancy loss (RPL) refers to habitual abortion that occurs 2~3 times or more miscarriage before the 20th weeks of gestation (Kim, M.S., Gu, B.H., Song, S., Choi, B.C., Cha, D.H., and Baek, K.H. ITI-H4, as a biomarker in the serum of recurrent pregnancy loss patients. Mol. BioSyst. 2011, 7). Although recurrent pregnancy loss is derived from the interaction of various factors, such as fetal or parent’s chromosomal abnormalities, uterine anatomic abnormalities, hormone secretion abnormalities, immunological abnormalities, etc., the causes of 40 to 50% of recurrent pregnancy losses were not found (Li, L.; Choi, B.C.; Ryoo, J.E.; Song, S.J.; Pei, C.Z.; Lee, K.Y.; Paek, J.; Baek, K.H. Opposing roles of inter-alpha-trypsin inhibitor heavy chain 4 in recurrent pregnancy loss. EBioMedicine 2018, 37). Therefore, there is no method for preventing and diagnosing recurrent pregnancy loss; and there is no specific therapeutic method thereto.

As a diagnostic method of recurrent pregnancy loss, amniocentesis tests, umbilical cord blood tests, and chorionic biopsies are currently available. However, since these methods may cause complications such as miscarriage, many patients are refusing the tests and thus there is a lot of resistance in clinical applications. Therefore, there is a need in the art for development and function-identification of the biomarkers that can easily diagnose recurrent pregnancy loss through blood tests, etc.

### DISCLOSURE

### Technical Problem

The present inventors have performed various searches for the genes whose expressions are specifically changed in recurrent pregnancy loss patients. As the results thereof, the present inventors have found that the expression of HtrA4 protein in recurrent pregnancy loss patients is significantly lower, in comparison with that in a normal person; and revealed the function of HtrA4 protein in recurrent pregnancy loss. Therefore, detection of the low expression level of HtrA4 protein can be usefully used to diagnose recurrent pregnancy loss; and the HtrA4 protein or a gene encoding the same can be used as a biomarker for diagnosing recurrent pregnancy loss.

Accordingly, it is an object of the present invention to provide an analytical method for providing information necessary for diagnosing recurrent pregnancy loss, using HtrA4 protein or a gene encoding the same.

It is another object of the present invention to provide a kit for diagnosing recurrent pregnancy loss, comprising a molecule capable of measuring the expression level of HtrA4 protein or a gene encoding the same.

### Technical Solution

In accordance with an aspect of the present invention, there is provided an analytical method for providing information necessary for diagnosing recurrent pregnancy loss, comprising measuring an expression level of HtrA4 protein or an expression level of a gene encoding the same in a subject’s sample.

In the analytical method of the present invention, the subject’s sample may be blood or serum. The measuring an expression level of HtrA4 protein may be carried out by Western blotting or enzyme-linked immunosorbent assay (ELISA). The measuring an expression level of a gene encoding HtrA4 protein may be carried out by measuring an amount of mRNA, preferably by measuring an amount of mRNA through RT-PCR (Reverse Transcription PCR) or real-time PCR.

In accordance with another aspect of the present invention, there is provided a kit for diagnosing recurrent pregnancy loss, comprising a molecule capable of measuring an expression level of HtrA4 protein or an expression level of a gene encoding the same, wherein the molecule is an antibody, a substrate, a ligand, or a cofactor, which specifically binds to the protein; or a primer having a complementary sequence specific to the gene encoding the protein.

In the diagnostic kit of the present invention, the molecule may be labeled with a detectable label. In another embodiment, the kit may be in the form of a microarray in which the primer is immobilized on a substrate.

### ADVANTAGEOUS EFFECTS

It has been found by the present invention that the expression of HtrA4 protein in recurrent pregnancy loss patients is significantly lower, in comparison with that in a normal person. Therefore, the analytical method and kit according to the present invention can be usefully applied for diagnosing recurrent pregnancy loss. That is, the HtrA4 protein or a gene encoding the same can be usefully applied as a biomarker for diagnosing recurrent pregnancy loss.

### DESCRIPTION OF DRAWINGS

FIG. 1 shows the results obtained by measuring the expression levels of HtrA4 in each serum derived from the recurrent pregnancy loss patients and the normal control group. (C: the normal group (the control group), P: recurrent pregnancy loss patients)

FIG. 2 shows the results of statistical analysis on the relative expression ratio of HtrA4, based on the results of FIG. 1.

FIG. 3 shows the results obtained by measuring the expression levels of HtrA4 in the choriocarcinoma cell lines, i.e., BeWo, HTR/SVneo cells, and JEG3 cells, through Western blotting analysis.

FIG. 4 shows the results obtained by confirming the knockout through T7E1 analysis in the two BeWo cell lines having knockout of the HtrA4 gene.

FIG. 5 is the results obtained by performing TA-cloning on knockout positions of the HtrA4 gene in the wild-type and knockout BeWo cell lines and then performing the sequence analyses thereof.

FIG. 6 shows the results obtained by measuring the expression levels of HtrA4 in the wild-type and knockout BeWo cells through Western blotting analysis.

FIG. 7 show the results obtained by measuring proliferation abilities of the wild-type and knockout BeWo cells, using CCK-8 assay.

FIG. 8 shows the results obtained by measuring proliferation abilities of the wild-type and knockout BeWo cells through colony formation analysis.

FIG. 9 shows the results obtained by measuring the expression levels of ERK, p-ERK, p38, and p-p38, which are related to cell proliferation, in the wild-type and knockout BeWo cells, through Western blotting analysis.

FIG. 10 shows the results obtained by the statistical analysis on relative protein expression ratios of ERK, p-ERK, p38, and p-p38, based on the results of FIG. 9.

FIG. 11 shows the results obtained by measuring the expression levels of Cyclin A and Cyclin E, which regulate the cycle of cells, in the wild-type and knockout BeWo cells through Western blotting analysis.

FIG. 12 shows the results obtained by the statistical analysis on relative protein expression ratios of Cyclin A and Cyclin E, based on the results of FIG. 11.

FIG. 13 shows the results obtained by measuring invasion abilities of the wild-type and knockout BeWo cells through invasion assay.

FIG. 14 shows the results obtained by measuring the expression levels of MMP-2 and MMP-9, which are associated with the invasive abilities of the wild-type and knockout BeWo cells, through Western blotting analysis.

FIG. 15 shows the results obtained by the statistical analysis on relative protein expression ratios of MMP-2 and MMP-9, based on the results of FIG. 14.

FIG. 16 shows the results obtained by measuring migration abilities of the wild-type and knockout BeWo cells through scratch wound assay.

FIG. 17 shows the results obtained by measuring the expression level of FAK associated with cell-migration ability in the wild-type and knockout cells, though Western blotting analysis.

FIG. 18 shows the results obtained by the statistical analysis on relative protein expression ratios of FAK, based on the results of FIG. 17.

FIG. 19 shows the photographs showing morphologies of the wild-type and knockout cells, at different magnifications, with a microscope.

FIG. 20 shows the results obtained by measuring the expression level of VE-cadherin, which is a factor promoting cell-to-cell connection in the wild-type and knockout cells, through Western blotting analysis.

FIG. 21 shows the results obtained by the statistical analysis on relative protein expression ratio of VE-cadherin, based on the results of FIG. 20.

### BEST MODE FOR CARRYING OUT THE INVENTION

As used herein, the term “recurrent pregnancy loss” refers to a disease of at least 2 or 3 consecutive miscarriage prior to the 20th weeks of gestation.

The present inventors have performed various searches for the genes whose expressions are specifically changed with respect to the serum samples derived from recurrent pregnancy loss patients and a normal group. As the results thereof, the present inventors have found that the expression of the specific protein (i.e., HtrA4 protein) in recurrent pregnancy loss patients is significantly lower, in comparison with that in a normal person. And, in order to investigate the mechanism of the differential expression of HtrA4 protein on recurrent pregnancy loss, the present inventors selected the BeWo cell line showing a high expression level of HtrA4, established the HtrA4 gene-knockout cells, and evaluated the effects of HtrA4 on the proliferation, migration, invasion, adhesion, etc., of cells, through the comparison of the wild-type and knockout cell lines. As the results thereof, the present inventors have found that the invasion and adhesion functions are more active in the wild-type BeWo cells than in the knockout BeWo cells, while the proliferation and migration functions are more active in the knockout BeWo cells than in the wild-type BeWo cells; and that the cell cycle is shorter in HtrA4 knockout BeWo cells than the wild-type BeWo cells. From these results, we demonstrated that the HtrA4 protein plays an important role in embryo implantation, placental formation, embryonic development and functions. Thus, the HtrA4 protein and the gene encoding the same can be usefully applied as a biomarker for the prediction of recurrent pregnancy loss and for diagnostic kit thereof.

The present invention provides an analytical method for providing information necessary for diagnosing recurrent pregnancy loss, comprising measuring an expression level of HtrA4 protein or an expression level of a gene encoding the same in a subject’s sample.

The HtrA4 protein and the gene encoding the same used as a biomarker in the analytical method of the present invention are known in the art, and thus the known protein(s) and gene sequence(s) can be used in the analytical method of the present invention. That is, the NCBI accession numbers of the HtrA4 (HtrA serine peptidase 4) protein are NP_710159.1, XP_011542733.1, and XP_011542734.1 and the NCBI accession numbers of the mRNA encoding the same are NM_153692.4, XM_011544431.2, XM_011544432.1, and BC057765.1.

In the analytical method of the present invention, the subject’s sample refers to a sample externally discharged from the human body, including e.g., blood, serum, etc. externally discharged from the human body.

The analytical method of the present invention includes measuring an expression level of HtrA4 protein or an expression level of a gene encoding the same. The amino acid sequence of HtrA4 protein and the genetic sequence encoding the same may be sequences known in GenBank as described above.

The measuring an expression level of HtrA4 protein or the gene thereof may be carried out by measuring a level of the protein or an mRNA of the gene, according to a method conventionally used in the field of biotechnology.

The measuring an expression level of HtrA4 protein may be carried out by Western blotting or enzyme-linked immunosorbent assay. For example, when the expression level of HTR4 protein is significantly lower than that of a normal person (e.g., at least about 4 times lower), the subject may be determined as a patient having risk of recurrent pregnancy loss. And, the measuring an expression level of a gene encoding HtrA4 protein may be carried out by measuring an amount of mRNA, preferably by measuring an amount of mRNA through RT-PCR or real-time PCR. When the expression level of HTR4 gene is significantly lower than that of a normal person, the subject may be determined as a patient having risk of recurrent pregnancy loss.

The present invention also provides a kit for diagnosing recurrent pregnancy loss, comprising a molecule capable of measuring an expression level of HtrA4 protein or an expression level of a gene encoding the same, wherein the molecule is an antibody, a substrate, a ligand, or a cofactor, which specifically binds to the protein; or a primer having a complementary sequence specific to the gene encoding the protein.

In the kit of the present invention, the HtrA4 protein may be used to prepare a polyclonal antibody or a monoclonal antibody; and a diagnostic kit comprising the antibody may be also prepared, according to a method conventionally used in the field of biotechnology. And, since the function of the HtrA4 protein has been revealed, the kit of the present invention may be prepared to comprise a substrate, a ligand, or a cofactor thereto. In addition, a primer having a complementary sequence specific to the gene encoding the protein may be prepared and a diagnostic kit comprising the primer may be also prepared, according to a method conventionally used in the field of biotechnology.

In the diagnostic kit of the present invention, the molecule capable of measuring an expression level of a gene encoding said protein may be labeled with a detectable label (e.g., a chromophore, etc.). And, the diagnostic kit of the present invention may be in the form of a microarray, e.g., in the form of a chip such as a DNA chip or a protein chip, in which the primer is immobilized on a substrate.

Hereinafter, the present invention will be described more specifically by the following examples. However, the following examples are provided only for illustrations and thus the present invention is not limited to or by them.

### EXAMPLES

### 1. Test Methods

### Preparation of Samples

This study was authorized by the Ethics Committee of CHA University located in Seoul, Korea. All participants in the study signed an informed consent and this study with human blood samples was authorized by an Institutional Review Board (Reference Number: 08-16). Human blood was collected from recurrent pregnancy loss patients (60) and normal groups (32) who visited the Fertility Center of the CHA General Hospital. Blood was divided into peripheral blood mononuclear cells (PBMC) and sera, and only sera were used in this study. The sera were stored at -80° C. The diagnostic criteria for recurrent pregnancy loss patients was based on the women having a documented history at least 2 or3 spontaneous and consecutive miscarriages prior to the 20th weeks of gestation. The normal group (the control group) was based on the women without obstetric complications, with at least one birth and not a history of abortion.

### Establishing Knockout of HtrA4 Gene in BeWo Cells

sgRNA was designed from the homepage (http://www.rgenome.net) [forward primer: 5′-CAG CGG CAC AGG TCG AAC ACC GG-3′ (SEQ ID NO: 1) and reverse primer: 5′-AAA CCA GAC TTC ACG CTC GGC-3′ (SEQ ID NO: 2)]. The primers were cloned to the pSpCas9(BB)-2A-GFP (pX458) vector (Addgene, Plasmid, Catalog #: 48138) to produce a sgRNA-pX458 plasmid. The sgRNA-pX458 plasmid was transfected into the choriocarcinoma cell line, i.e., BeWo cells (KCLB:10098, Korean Cell Line Bank) by electroporation using Lonza 4D-Nucleofector (Lonza, 50829 Cologne, Germany) according to the manufacturer’s instructions. The resulting cells were seeded into 96-well plates with 1 cell/well and incubated for 8 days to reach 60%-70% confluent. Genomic DNAs were extracted using Accuprep Genomic DNA Extraction Kit (Bioneer Corporation, Daejeon, Korea) according to the manufacturer’s instructions. Polymerase chain reaction (PCR) was carried out using the forward primer: 5′-GAG GGT TTG CAG GTC CAG AG-3′ (SEQ ID NO: 3) and the reverse primer: 5′-ACA TGC TGG GGT AGG TGC-3′ (SEQ ID NO: 4). T7E1 assay for the PCR products was performed for selecting the cells in which the HtrA4 gene was knocked out. TA cloning was performed to confirm the base sequences thereof. The expression level of HtrA4 was confirmed through Western blotting analysis.

### Colony Forming Assay

The wild-type and knockout cell lines were seeded with the same number, and then stained with crystal violet for colony forming assay. The cells were seeded into a 96-well plate with 1 × 10³ cell/well and then incubated for 0, 24, 36, 48, and 60 hours. The cells were treated with DMEM (Dulbecco’s Modified Eagle Medium) containing 10 µl of WST-8 solution (Cell Counting Kit-8 Dojindo, Kumamoto, Japan) and then incubated for 4 hours. Subsequently, the OD values were measured at 450 nm. After 14 days, the proliferation ability of cells was confirmed by comparing the number of colonies formed by the wild-type and the knockout cells.

### Scratch Wound Assay

The wild-type and knockout cell lines were seeded into a 6-well plate. When the cells were cultured until 70-80% confluence, they were scraped with a micropipette tip so as to cause damages thereto. Using the PBS, the cells were washed for removing stripped cells. The photographs were obtained at 0, 24, and 48 hours to analyze using the Image-J software. The digital photographs were obtained using a microscope (Olympus, Tokyo, Japan).

### Invasion Assay

The wild-type and knockout cell lines were seeded into the 24-well upper chamber (80-µm pore membranes; BD Biosciences, Franklin Lakes, NJ, USA) with the serum-free medium (DMEM) at a density of 1×10⁶ cell/ml. The DMEM containing 10% FBS was added into the lower chamber. After being incubated for 36 hours at 37° C., the non-invasive cells were removed from the upper chamber using cotton swabs. The cells in the lower membrane surface were stained with crystal violet for 10 seconds to attach the invasive BeWo cells. After the invasive chamber was washed twice by phosphate-buffered saline (PBS), the number of invasive BeWo cells was counted under a microscope (Olympus, Tokyo, Japan). Each experiment was performed in triplicate.

### Western Blotting Analysis

The wild-type and knockout cells were dissolved in a lysis buffer on ice for 20 minutes, respectively, and then centrifuged at 13,000 rpm for 20 minutes. The samples were boiled with 2X SDS protein loading buffer for 10 minutes, loaded into SDS-PAGE gel, and then the proteins were transferred onto polyvinylidene fluoride (PVDF) microporous membrane (Millipore, Billerica, MA, USA). The membrane was incubated with a primary antibody at 4° C. overnight, washed, and then incubated with a secondary antibody at room temperature for 1 hour. The blots were detected using the ECL reagent solution (Young In Frontier, Seoul, Korea).

### Antibodies

The anti-HtrA4 antibody was purchased from Proteintech (Proteintech, Rosemont, IL, USA). The anti-ERK1/2, anti-p-ERK (Thr202/Tyr204), anti-p38, anti-p-p38, anti-Cyclin A, anti-Cyclin E, and anti-VE-cadherin antibodies were purchased from Cell Signaling Technology (Cell Signaling Technology Inc., Danvers, MA, USA). The anti-MMP-2, anti-MMP-9, anti-p-FAK (Try397), and anti-β-actin antibodies were purchased from Santa Cruz Biotechnology (Santa Cruz Biotechnology, CA, USA).

### Analysis of the Derived Results

The densitometric analysis was performed with Image J (National Institutes of Health, Bethesda, MD, USA) and the t-test was performed with GraphPad Prism version 5 (GraphPad Software, La Jolla, CA, USA). ANOVA was performed by one-way analysis to indicate significant differences.

### 2. Test Results

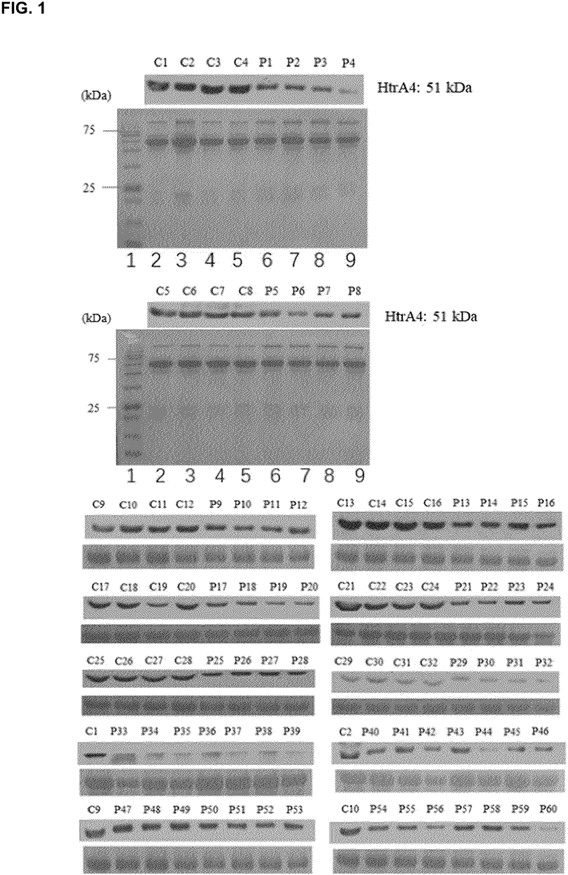

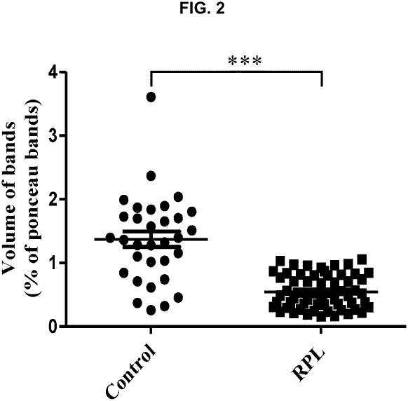

FIGS. 1 and 2 show the results obtained by measuring the expression levels of HtrA4 in the serum through Western blotting analysis, after the blood of the recurrent pregnancy loss patients and the normal control group were separated into sera and PBMCs. FIG. 1 is the results obtained by measuring the expression levels of HtrA4 in each serum derived from the recurrent pregnancy loss patients and the normal control group. FIG. 2 is the results of statistical analysis on the relative protein expression ratio of HtrA4, based on the results of FIG. 1. From the results of FIG. 1 and FIG. 2, it can be confirmed that HtrA4 is expressed at significantly lower levels in the recurrent pregnancy loss patients.

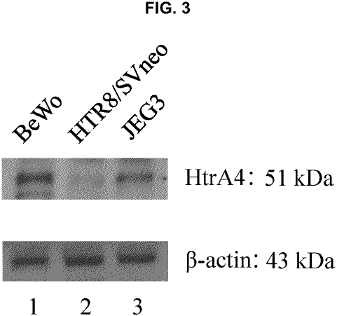

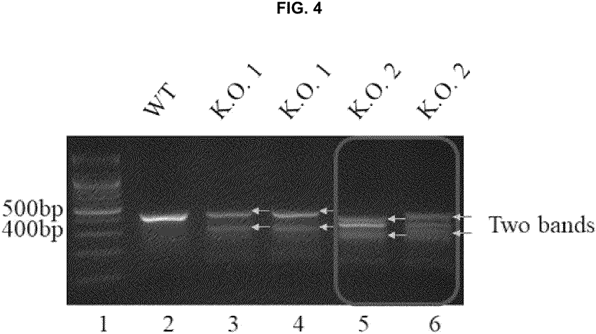

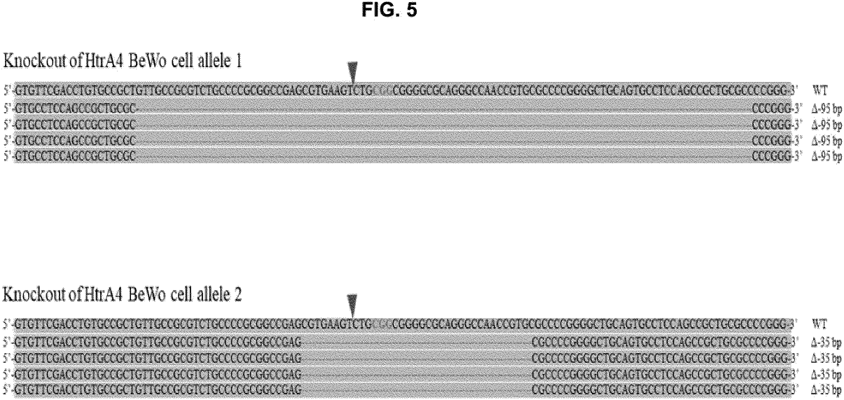

FIG. 3 shows the results obtained by measuring the expression levels of HtrA4 in the choriocarcinoma cell lines, i.e., BeWo, HTR/SVneo, and JEG3 cells, which confirm that the expression level of HtrA4 is the highest in the BeWo cells. FIG. 4 shows the results obtained by confirming the knockout through T7E1 analysis in the two BeWo cell lines having knockout of the HtrA4 gene and the wild-type BeWo cell line. Both K.O.1 and K.O.2 cell lines show knockout of the HtrA4 gene; and two alleles were knocked out in K.O.2. FIG. 5 is the results obtained by performing TA cloning of the two bands shown in the K.O.2 cell line and then performing the sequence analyses thereof. We confirmed that Δ95 bp was knocked out in allele 1 and that Δ35 bp was knocked out in allele 2. The subsequent experiments were carried out using the K.O.2 cell line.

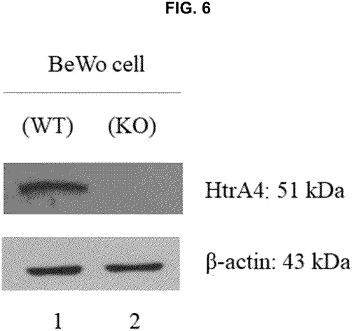

FIG. 6 is the results obtained by measuring the expression levels of HtrA4 in the wild-type and knockout BeWo cells through Western blotting analysis. It can be confirmed that the expression of HtrA4 in the knockout cells is significantly lower.

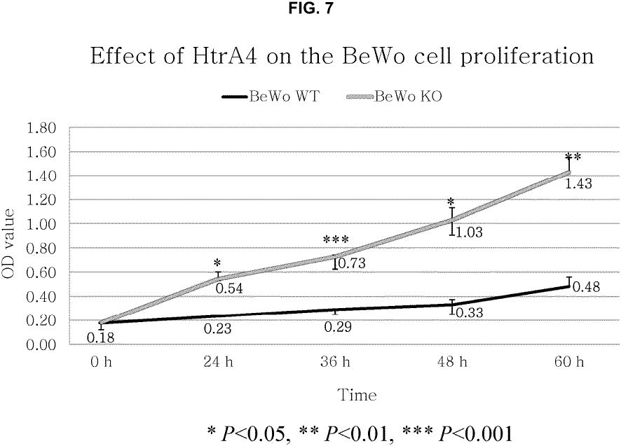

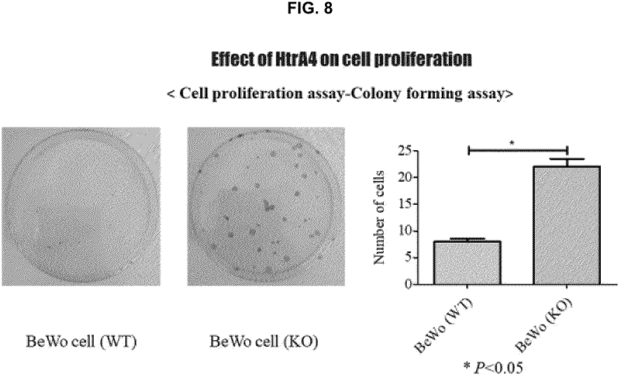

FIG. 7 is the results obtained by measuring proliferation abilities of the wild-type and knockout BeWo cells, using the CCK-8. It can be confirmed that the knockout cells show statistically significant proliferation ability after 24 hours and that the proliferation was faster over time. FIG. 8 is the results obtained by measuring proliferation abilities of the wild-type and knockout BeWo cells, through colony formation analysis. It can be confirmed that the proliferation ability of the knockout cells is stronger as expected.

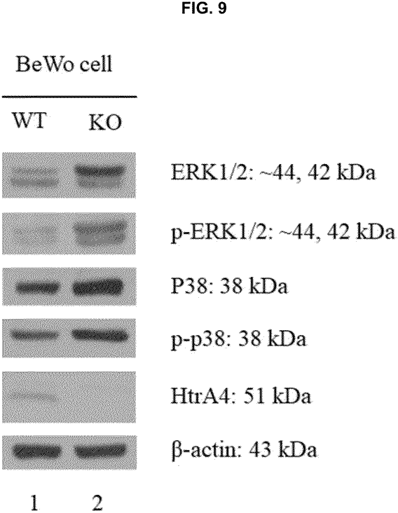

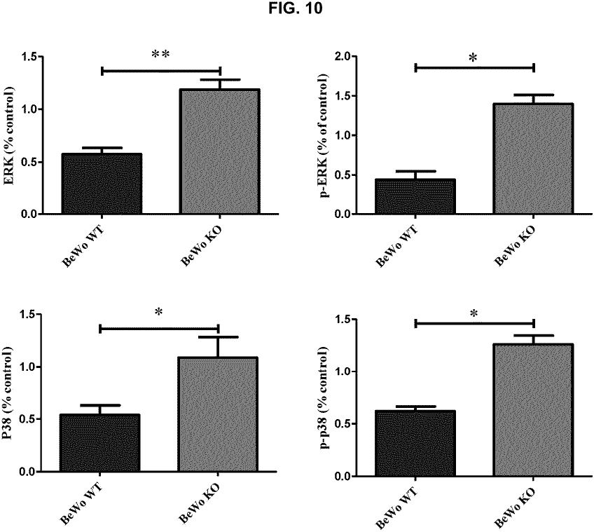

FIG. 9 is the results obtained by measuring the expression levels of ERK, p-ERK, p38, and p-p38 which are related to cell proliferation, in the wild-type and knockout BeWo cells. FIG. 10 is the results obtained by the statistical analysis on relative protein expression ratios of ERK, p-ERK, p38, and p-p38, based on the results of FIG. 9. From the results of FIGS. 9 and 10, it can be confirmed that the knockout cells having stronger cell proliferation ability show higher expressions of the ERK, p-ERK, p38, and p-p38 which promote the proliferation of cells.

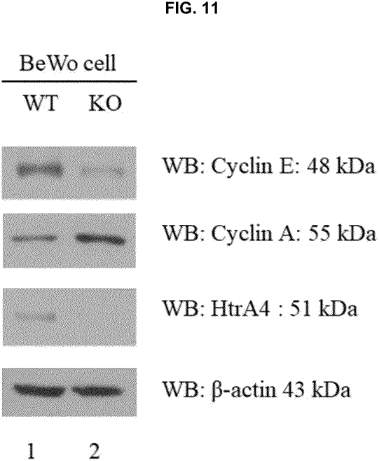

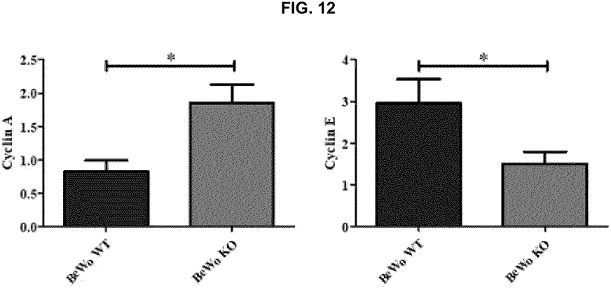

FIG. 11 is the results obtained by measuring the expression levels of Cyclin A and Cyclin E, which regulate the cycle of cells, through Western blotting analysis. FIG. 12 is the results obtained by the statistical analysis on relative protein expression ratios of Cyclin A and Cyclin E, based on the results of FIG. 11. From the results of FIGS. 11 and 12, it can be confirmed that the wild-type BeWo cells show the higher expression level of the Cyclin E which is associated in conversion from G1 phase to S phase, while the knockout cells show the higher expression level of the Cyclin A which is associated in conversion from S phase to G2 phase.

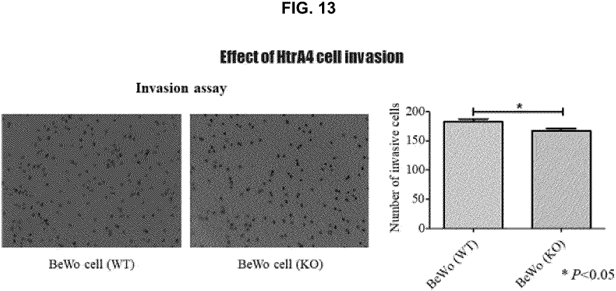

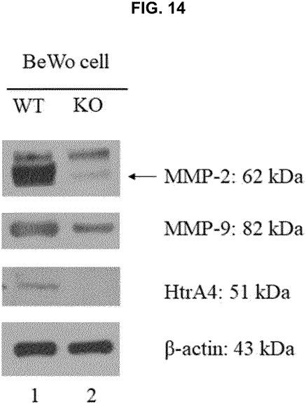

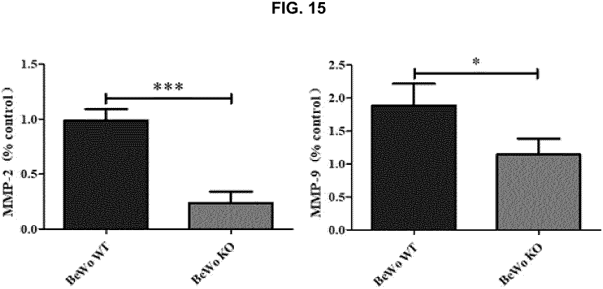

FIG. 13 is the results obtained by measuring invasion abilities of the wild-type and knockout BeWo cells through invasion assay. It can be confirmed that the invasive ability of the knockout cells is weaker than that of the wild-type cell. FIG. 14 is the results obtained by measuring the expression levels of MMP-2 and MMP-9, which are associated with the invasive abilities of the wild-type and knockout BeWo cells, through Western blotting analysis. FIG. 15 is the results obtained by the statistical analysis on relative protein expression ratios of MMP-2 and MMP-9, based on the results of FIG. 14. From the results of FIGS. 14 and 15, it can be confirmed that the knockout cells show lower expression levels of the MMP-2 and MMP-9 which promote the invasion of cells.

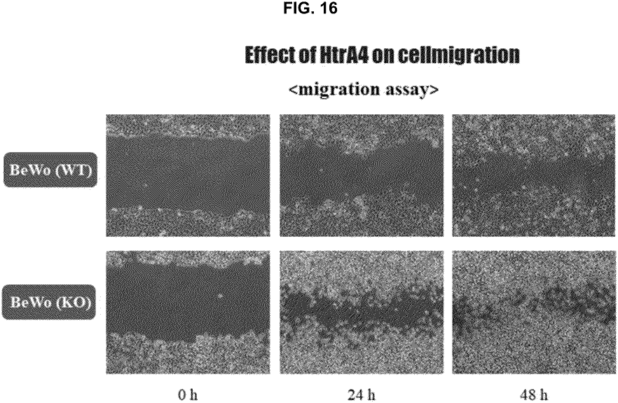

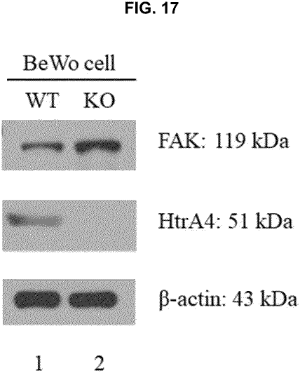

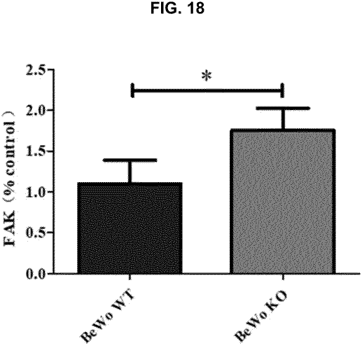

FIG. 16 is the results obtained by measuring migration abilities of the wild-type and knockout BeWo cells through scratch wound assay. It can be confirmed that the knockout cells show stronger migration ability than the wild-type cells. FIG. 17 is the results obtained by measuring the expression level of FAK associated with cell-migration ability in the wild-type and knockout cells. FIG. 18 is the results obtained by the statistical analysis on relative protein expression ratios of FAK, based on the results of FIG. 17. From the results of FIGS. 17 and 18, it can be confirmed that the knockout cells having stronger migration ability show the higher expression of the FAK which promotes cell-migration, than the wild-type cells.

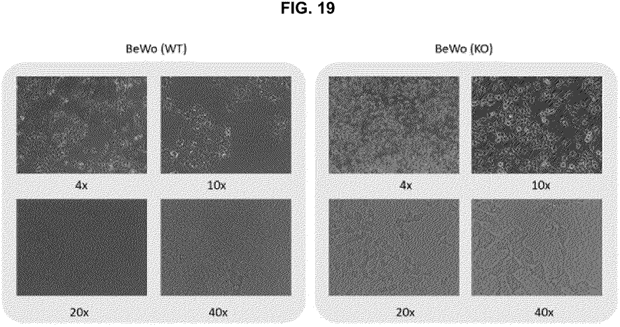

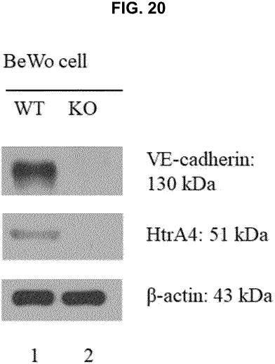

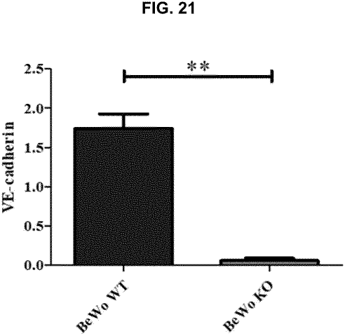

FIG. 19 is the photographs showing morphologies of the wild-type and knockout cells, at different magnifications, under a microscope. As can be confirmed in FIG. 19, the wild-type cells grow in close contact with each other, while the knockout cells grow without tight contact therewith. FIG. 20 is the results obtained by measuring the expression level of VE-cadherin, which is a factor promoting cell-to-cell connection in the wild-type and knockout cells, through Western blotting analysis. FIG. 21 is the results obtained by the statistical analysis on relative protein expression ratio of VE-cadherin, based on the results of FIG. 20. As expected, it can be confirmed that the knockout cells show lower expression of the VE-cadherin which promotes cell-to-cell connection, than the wild-type cells.

### 3. Discussion

Although there have been carried out many studies on the causes of recurrent pregnancy loss, the researches on pathogenesis, e.g., proteases, of recurrent pregnancy loss are still insufficient. The present inventors have found that the HtrA4 gene is expressed at a lower level in the sera of recurrent pregnancy loss patients, in comparison with a normal group. This demonstrates that HtrA4 is a biomarker of recurrent pregnancy loss and that HtrA4 can be utilized in the manufacture of a kit capable of predicting and diagnosing recurrent pregnancy loss, through using a small amount of blood.

Based on these results, the present inventors carried out the studies on the function of the HtrA4 gene in recurrent pregnancy loss. For the studies, the present inventors established the BeWo cell line in which the HtrA4 gene is knocked out. The HtrA4 knockout BeWo cell line was compared with the wild-type BeWo cells. As the results thereof, the present inventors found that HtrA4 promotes invasion and adhesion of the placental chorionic cells and inhibits proliferation and migration of the cells. Therefore, the present inventors demonstrate that the low expression level of HtrA4 in recurrent pregnancy loss patients affects the placental chorionic cell functions and causes abnormalities in the formation, growth and function of the placenta, which results in miscarriage.

1. An analytical method for providing information necessary for diagnosing recurrent pregnancy loss, comprising measuring an expression level of HtrA4 protein or an expression level of a gene encoding the same in a subject’s sample.

2. The analytical method according to claim 1, wherein the subject’s sample is a blood or serum sample.

3. The analytical method according to claim 1, wherein the measuring an expression level of HtrA4 protein is carried out by Western blotting or enzyme-linked immunosorbent assay.

4. The analytical method according to claim 1, wherein the measuring an expression level of a gene encoding HtrA4 protein is carried out by measuring an amount of mRNA.

5. The analytical method according to claim 4, wherein the measuring an amount of mRNA is carried out by RT-PCR or real-time PCR.

6. A kit for diagnosing recurrent pregnancy loss, comprising a molecule capable of measuring an expression level of HtrA4 protein or an expression level of a gene encoding the same, wherein the molecule is an antibody, a substrate, a ligand, or a cofactor, which specifically binds to the protein; or a primer having a complementary sequence specific to the gene encoding the protein.

7. The kit according to claim 6, wherein the molecule is labeled with a detectable label.

8. The kit according to claim 6, wherein the kit is in the form of a microarray in which the primer is immobilized on a substrate.
